(19) United States
(12) Patent Application Publication
(10) Pub. No.: US 2024/0277645 A1
(43) Pub. Date: Aug. 22, 2024

(54) CONTINOUS IMPREGNATION OF ACTIVE PHARMACEUTICAL INGREDIENTS ONTO POROUS CARRIERS

(71) Applicants: MERCK PATENT GMBH, DARMSTADT (DE); RUTGERS, THE STATE UNIVERSITY OF NEW JERSEY, New Brunswick, NJ (US)

(72) Inventors: Fernando J. MUZZIO, Long Branch, NJ (US); Benjamin J. GLASSER, Princeton, NJ (US); Thamer A. OMAR, Piscataway, NJ (US); Andrés D. ROMÁN-OSPINO, Newton, MA (US); Gudrun BIRK, Darmstadt (DE)

(73) Assignees: MERCK PATENT GMBH, DARMSTADT (DE); RUTGERS, THE STATE UNIVERSITY OF NEW JERSEY, New Brunswick,, NJ (US)

(21) Appl. No.: 18/571,083

(22) PCT Filed: Jun. 13, 2022
(86) PCT No.: PCT/US2022/033183

§ 371 Date: Dec. 15, 2023

Related U.S. Application Data
(60) Provisional application No. 63/210,700, filed on Jun. 15, 2021.

(51) Int. Cl. A61K 31/192; A61K 9/14; A61K 9/16; A61K 9/48; A61K 31/55

(52) U.S. Cl. CPC A61K 31/192; A61K 9/143; A61K 9/1611; A61K 9/4825; A61K 31/55

(57) ABSTRACT

The disclosed technology relates to a continuous impregnating process of active pharmaceutical ingredients (API) onto porous carriers, a continuous impregnation process for making impregnated porous carrier particles and pharmaceutical dosage forms comprising impregnated porous carrier particles, impregnated porous carrier particles and pharmaceutical dosage forms comprising impregnated porous carrier particles prepared by the continuous impregnation process.

### TECHNICAL FIELD

The disclosed technology relates to a continuous impregnating process of active pharmaceutical ingredients (API) onto porous carriers, a continuous impregnation process for making impregnated porous carrier particles and pharmaceutical dosage forms comprising impregnated porous carrier particles, impregnated porous carrier particles and pharmaceutical dosage forms comprising impregnated porous carrier particles prepared by the continuous impregnation process.

### BACKGROUND

Impregnation of active pharmaceutical ingredients onto porous carriers is increasingly of interest for addressing drugs that exhibit poor water solubility. In this technology, the poorly water-soluble drug substance is dissolved in a volatile organic solvent and is impregnated inside a porous carrier. Solvent is subsequently evaporated, leaving the drug substance deposited on the surface of the pores inside the carrier. Typically, the process is conducted so that the drug is deposited in a thin, amorphous layer that dissolves rapidly when the impregnated carrier is subsequently exposed to dissolution media. Methods for impregnating a porous carrier with an active pharmaceutical ingredient have been described previously.

WO 2012/027222 discloses methods for impregnating a porous carrier with an active pharmaceutical ingredient, the methods comprising steps: a) dissolving at least one API in a solvent to form an API solution; b) contacting a porous carrier with the at least one API of step (a) in a contactor to form an API-impregnated porous carrier; and c) drying the at least one API-impregnated porous carrier.

Impregnated porous carriers can be manufactured either using a batch or a continuous process. However, a problem that remains unsolved impregnating porous carriers continuously is how to achieve a high drug loading in a porous carrier, which is needed to create high dose products without requiring a large amount of carrier as to make the final product difficult to swallow. While it is possible to increase the drug loading by multi-pass impregnation of conventional porous carriers, it was observed that solubility enhancement decreases for API loadings beyond about 10%. This is problematic for high dose products (i.e., products where the unit dose contains 200 mg of API or more) because it requires large amounts of carrier, resulting in a very large unit dose that would be difficult to swallow.

Dissolution enhancement at higher API loadings, i.e, higher than 20%, can be archieved by using carriers with large surface area (greater than 400 m²/g). Those carriers typically have a small pore size and small particle size (D50 below 30 microns). Such materials are cohesive and very difficult to fluidize. Impregnation only has been shown to be successful when performed in batch mode using mechanically agitated vessels. Under such process conditions, homogeneous impregnation of such high surface area porous carriers would require contact times of one hour or more. The process time, which is limited by the time required to mix the carrier with the impregnation fluid, would increase significantly as the process is scaled up.

A problem to be solved is therefore the provision of an impregnation process, in particular a continuous impregnation process, that can archieve high API loadings with homogeneous impregnation (high content uniformity), preferably in a short contact time.

A further problem to be solved is the provision of an impregnation process, in particular a continuous impregnation process, that can archieve high API loadings (more than 10%) with an improved dissolution profile of the API.

A further problem to be solved is the provision of an impregnation process, in particular a continuous impregnation process, that can archieve a consistent particle size distribution before and after the impregnation steps.

### SUMMARY OF THE INVENTION

In the instant invention, it has been surprisingly found that high surface area porous carriers can be uniformly and homogeneously impregnated within only a few minutes of contact time between the carrier and the impregnation fluid when a continuous impregantaion process is used. A continuous impregnation process with high surface area porous carriers is particularly suitable for improving the API loading, in particular compared to a batch process or to a continuous impregnation process with different porous carriers within a defined process time. Furthermore, the content uniformity of the resulting impregnated porous carrier particles is surprisingly improved, in particular compared to porous carrier with a lower surface within a defined process time.

Unexpectedly, it was shown that a continuous impregnation process for making impregnated porous carrier particles which comprises the steps of continuously dispensing a solution containing at least one API and feeding a porous carrier in a controlled ratio into a continuous impregnation device, continuously mixing and conveying the drug solution and the porous carrier in the continuous impregnation device and evaporating solvent to form impregnated porous carrier particles, is particular suitable for solving the above mentioned problems when a high surface areas porous carrier is used, in particular a porous carrier that has a specific surface area of at least 400 m²/g.

In another aspect, the disclosed technology using porous carriers have an average pore size of 1 to 10 nm and/or an average particle size of 5 μm to 150 μm. In another aspect, the porous carrier is based on silicon oxide or silicon dioxide.

In another aspect, the impregnated porous carrier particles can be optionally mixed with at least one pharmaceutical excipient to form a pharmaceutical composition. Subsequently, the impregnated porous carrier particles or the pharmaceutical compositions of the invention can be processed into a solid dosage form.

In another aspect, the disclosed technology relates to impregnated porous carrier particles, pharmaceutical compositions and solid dosage forms obtainable or obtained by the process as described herein.

### DETAILED DESCRIPTION OF THE INVENTION

The following discussion omits or only briefly describes conventional features of the disclosed technology that are apparent to those skilled in the art. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations. A person of ordinary skill in the art would know how to use the instant invention, in combination with routine experiments, to achieve other outcomes not specifically disclosed in the examples or the embodiments. Unless otherwise specifically defined herein, all terms are to be given their broadest possible interpretation including meanings implied from the specification as well as meanings understood by those skilled in the art and/or as defined in dictionaries, treatises, etc. Unless defined otherwise, all technical and scientific terms used herein have the same meaning as commonly understood by one of ordinary skill in the art in the field of the disclosed technology.

Additionally, methods, equipment, and materials similar or equivalent to those described herein can also be used in the practice or testing of the disclosed technology. The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

The present invention discloses a continuous impregnation process for making impregnated porous carrier particles, comprising the steps:
a. continuously dispensing a solution containing at least one API and feeding a porous carrier in a controlled ratio into a continuous impregnation device,
b. continuously mixing and conveying the drug solution and the porous carrier in the continuous impregnation device and
c. evaporating solvent to form impregnated porous carrier particles,
wherein the porous carrier has a specific surface area of at least 400 m²/g.

In general, the term “impregnation” is the process of placing chemical substances (such as APIs) inside porous carriers using a solution or a suspension that penetrates the pores of the carrier particles generating “impregnated porous carrier particles”. While not willing to be bound by theory, it is commonly observed that penetration of the solution or the suspension is aided by capillary action, so that favorable wetting conditions and lower solution/suspension viscosity leads to faster impregnation. The high surface area of porous carriers allows them to absorb several compounds, including materials that are poorly flowing in dry powder form, such as cohesive drugs. Since at the end of the process these compounds are completely (or nearly completely) embedded within the porous carriers, the flow and compaction properties of the impregnated products are very similar to those of the carrier, thereby facilitating their handling and further processing.

While the bulk density of the impregnated carrier will often be higher than that of the original porous carrier, obeying to the partial or complete filling of pores with API and other substances, this change in density usually does not affect the performance of the impregnated carrier in undesirable ways. Moreover, at least some of the drug within the pores of the carrier typically exists in an amorphous form, which improve its dissolution. Drug in amorphous form are known in the art and often used for the purpose of improving drug dissolution. In some cases, such amorphous drugs are physically unstable and exhibit a change in crystalline form over time, which can also result in changes in drug dissolution over the product shelf life. Improving the physical stability of such drug substances by porous carrier impregnation enhances the stability of important drug product properties, such as dissolution behavior. While not willing to be bound by theory, it is believed that the small sizes of many of the pores of the carrier result in confining of the drug, which limits the drug's mobility and its exposure to interstitial moisture, and also inhibits the drug's recrystallization inside the pores. Accordingly, impregnation into a porous carrier can improve crucial properties of the drug. Further, the disclosed process applies to impregnation using not only different APIs but also different porous carriers having a defined specific surface area, and the use of additional materials for the purpose of modulating drug dissolution, improving chemical and physical stability, and other desired features known to the skilled artisan.

“Continuous impregnation process” refers to a process that can be either a stand-alone process for manufacturing API-impregnated carrier, or can be part of a larger integrated continuous manufacturing line for manufacturing of pharmaceutical products containing API-impregnated carrier. Continuous manufacturing methods can provide significant technical and business advantages relative to batch methods. In general, continuous manufacturing methods are more robust and controllable. They achieve the same production rates as batch processes in much smaller and thus less capital-intensive equipment, which also requires less space to operate. The continuous manufacturing processes described herein may include sensing and control capabilities, such that the process is continuously monitored by various sensors and controllers to maintain the continuous process and the resulting products within the desirable operating range of process parameters and product quality attributes. Measurements collected from sensors can be used in conjunction with controllers and actuators arranged in a closed loop system (under closed loop control), using feedback, feed forward, and other configurations to control the performance of the process and the quality of the manufactured products.

In general, the API impregnation process includes the steps of: dissolving or suspending at least one API in a solvent to form an API solution; contacting a porous carrier with the API solution in a contactor to form an API-impregnated porous carrier; and drying the API-impregnated porous carrier.

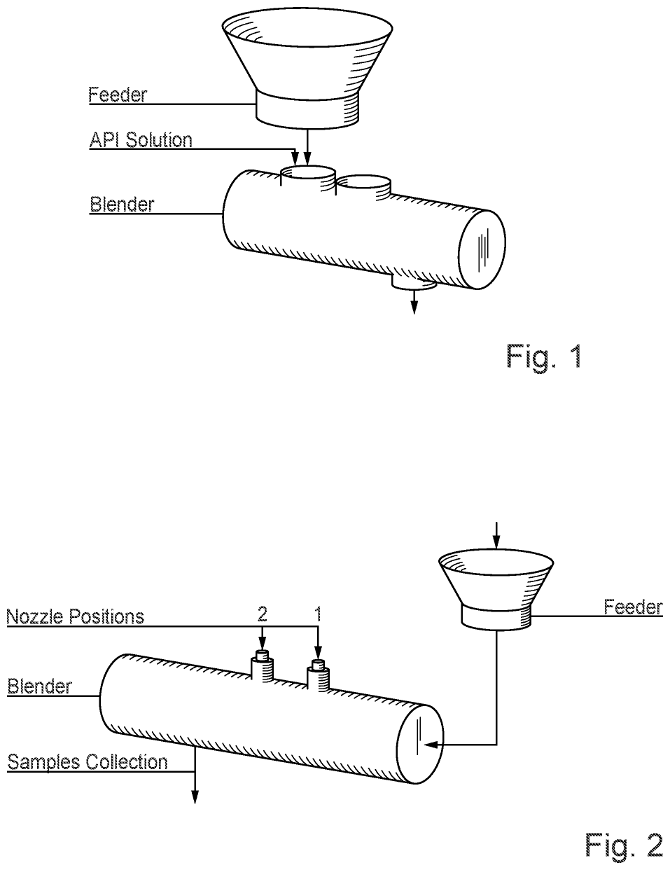

FIG. 1 shows an example of a portion of the disclosed continuous manufacturing process, wherein porous carrier is fed from a first feeder into a continuous impregnation device into which API solution is dispensed in order to form particles of API-impregnated porous carrier.

The solvent of the solution is a suitable volatile fluid that dissolves the at least one API and may be used to impregnate it into the carrier pores. The solvent may be an inorganic or organic liquid. Non-limiting examples of suitable liquids that may be used in the disclosed method include ethanol, methanol, isopropyl alcohol (IPA), acetone, 1-propanol, 1-pentanol, acetonitrile, butanol, methyl ethyl ketone (MEK), methyl acetate, 2-methyl tetrahydrofuran, isopropyl acetate (IPAc), n-hexane, ethyl acetate (EtOAc), n-heptane, water, an aqueous solvent, supercritical CO2, and combinations thereof. Surfactants, chelating agents, and other materials can be used as needed to enhance the dissolution in the solvent of poorly soluble drugs. The solution is dispensed on the porous carrier through at least one nozzle. The volume of dispensed API solution can be adjusted by the dispense rate that is calculated as the mass of the API solution being dispensed (kg) divided by the product of the total dispense time (s) and the mass of the carrier (kg). In some embodiments of the disclosed continuous manufacturing process, the API solution dispense rate is less than 1 s⁻¹, less than 0.5 s⁻¹, less than 0.4 s⁻¹, less than 0.3 s⁻¹ or less than 0.1 s⁻¹. In some embodiments, the concentration of API in the solvent may be in the range of 10⁻⁶ wt to 40 wt %, 10⁻⁶ wt to 30 wt %, 10⁻⁶ wt to 20 wt %, 10⁻⁶ wt to 10 wt %, 10⁻⁶ wt to 1 wt %, 10⁻⁵ wt to 40 wt %, 10⁻⁵ wt to 30 wt %, 10⁻⁵ wt to 20 wt %, 10⁻⁵ wt to 10 wt %, 10⁻⁵ wt to 1 wt %, 10⁻⁴ wt to 40 wt %, 10⁻⁴ wt to 30 wt %, 10⁻⁴ wt to 20 wt %, 10⁻⁴ wt to 10 wt %, 10⁻⁴ wt to 1 wt %, 10⁻³ wt to 40 wt %, 10⁻³ wt to 30 wt %, 10⁻³ wt to 20 wt %, 10⁻³ wt to 10 wt %, 10⁻³ wt to 1 wt %, 10⁻² wt to 40 wt %, 10⁻² wt to 30 wt %, 10⁻² wt to 20 wt %, 10⁻² wt to 10 wt %, 10⁻² wt to 1 wt %, 0.1 wt to 40 wt %, 15 0.1 wt to 30 wt %, 0.1 wt to 20 wt %, 0.1 wt to 10 wt %, 0.1 wt to 1 wt %, 1 wt to 40 wtl wt to 30 wt %, 1 wt to 20 wt %, 1 wt to 10 wt %, or 1 wt to 5 wt %. In all cases, “wt %” is calculated by dividing the amount of API in a sample or a batch by the total weight of the sample or the batch.

Any pharmaceutically suitable API may be used in the disclosed method. One or more different APIs (e.g., 1, 2, 3 or more APIs) may be impregnated into the porous carrier. In some embodiments, multiple APIs may be dissolved in solution in a fixed ratio (e.g., 1:1 to 3:1, 2:1, and variations thereof), after which the multi-API solution may be dispensed into the porous carrier to achieve API-impregnated porous carrier having the same fixed ratio of APIs. Specific APIs disclosed herein are provided for illustrative purposes only and do not limit the scope of the disclosed technology. In general, an API used in impregnation should possess three main properties: be stable under relevant experimental conditions, be soluble to a significant extent in different types of solvents, and be inert when combined with the porous carrier. In some embodiments, the API is suitably soluble in a volatile organic solvent. In some embodiments, the API is at least partially in amorphous form after impregnation. In some embodiments, the API is at least partially in crystalline form. In some embodiments, the API is suitably soluble in water, while in some other embodiments, the API is poorly soluble in water, e.g., the API solubility is less than 10 mg/ml. In yet other embodiments, the solubility of the API in water can be increased or decreased by modifying the pH of the solution. Yet in other embodiments, the pH-modifying substance is itself volatile (e.g., ammonia, CO2, and other such substances) such that it modifies the solubility of the API (or other substances present) during impregnation, but is largely eliminated by evaporation during the drying step of the process. Some non-limiting examples of APIs include acetaminophen, ibuprofen, carbamazepine, indometacin/indomethacin, flufenamic acid, imatinib, erlotinib hydrochloride, vitamin D, steroids, estrodial, other non-steroidal anti-inflammatory drugs (NSAIDs), and combinations thereof.

The first feeder is used to accurately dispense the porous carrier. From the first feeder, the porous carrier flows directly into a continuous impregnation device, where the porous carrier particles undergo agitation. The API impregnation step described above occurs in the continuous impregnation device into which API solution is dispensed. API-impregnated porous carrier particles then flow (e.g., fall by gravity) from the continuous impregnation device to a second, transitional feeder, which controls the bed height. An analytical instrument, such as a NIR probe, may be positioned above the transitional feeder to obtain spectral scans of the API-impregnated carrier particles passing underneath.

The term “continuous impregnation device” and “continuous blender” are used synonymously. The continuous impregnation device can take multiple forms (e.g., tubular mixer, vertical continuous impregnation device, zig zag mixer, continuous powder blender etc.) and may perform multiple operations within the continuous API impregnation process. In simple terms, impregnation includes two main steps: (1) mixing of API solution with a porous carrier, and (2) drying the resulting product. In the context of the disclosed process, agglomeration is undesirable and to be avoided, minimized or eliminated from the process. Agglomeration may occur when the dispense rate is high such that a liquid layer of API solution exist around the host carrier, which “glues” the particles to each other. Agglomeration or granulation may also occur when the impregnation ratio is too high, which allows a high amount of API solution to penetrate the pores, leading to pore saturation, accumulation of API solution at the surface of carriers and (undesirable) granulation.

In batch processes, impregnation is desirably achieved when Ri (impregnation rate)>Rd (drying rate)≥Rs (dispensing rate). This allows for both high penetration of API solution into the pores of the porous carrier and evaporation of solvent before saturation. In a continuous process, the drying step is subquent to the mixing of the solution with the carrier, and therefore the main condition required to avoid agglomeration is that Ri (impregnation rate)>Rs (dispensing rate).

FIG. 2 shows an example of a portion of the disclosed continuous manufacturing process, wherein the porous carrier is fed from a first feeder into a continuous impregnation device and API solution is simultaneously dispensed into the continuous impregnation device. The continuous impregnation device is supplied with at least one nozzle (e.g., one, two, three, four, or more nozzles) for dispensing of the API solution into the porous carrier. The nozzle(s) may be located at various positions along the axis of the blender, such as near the blender entrance (e.g., approximately 25% of the full length of the blender) and/or at or near the midpoint of the blender. The API solution may be directed to the nozzle(s) via tubing, such as Tygon® (flexible polymer) tubing. One or more nozzles may have perforations with a diameter of 0.05 mm to 0.5 mm, 0.1 mm to 0.4 mm, or 0.2 mm to 0.3 mm. A pump, such as a peristaltic pump, may be 20 utilized to deliver the API solution to the continuous impregnation device, e.g. from a source supply of API solution through the tubing connected to the one or more nozzles, and then into the blender.

In some embodiments, the API solution is delivered into the continuous impregnation device at a pumping rate of up to 1000 ml/min, such as 1 ml/min to 1000 ml/min, 10 ml/min to 800 ml/min, 20 ml/min to 600 ml/min, 30 ml/min to 500 ml/min, 20 ml/min to 400 ml/min, 55 ml/min to 300 ml/min, 85 ml/min to 200 ml/min, 25 ml/min to 100 ml/min, or 60 ml/min to 90 ml/min. Ingredients in the continuous impregnation device are mixed and impregnated simultaneously. The blender may be operated at various rotation speeds, such as 150-600 rpm, 150-300 rpm, 300-600 rpm, about 150 rpm, about 300 rpm, or about 600 rpm.

In some embodiments, the continuous impregnation device is a continuous tubular blender. In some embodiments, the continuous impregnation device includes multiple blades or paddles. For example, the blender may have a one-third forward-alternating-forward blade configuration, wherein a first set of paddles (e.g., 5-10 paddles or 6-8 paddles), an optionally equal last set of paddles are all angled in the forward direction to convey the powder forward through the process, and an optionally equal middle set of paddles may be angled in an alternating forward and backward direction, creating a zone of back-mixing. An alternating angle pattern of blades in the continuous impregnation device can create a region of material hold-up, which may be a desirable region into which the API solution is dispensed in order to more effectively dispense the API solution across the porous carrier material. API impregnation of the porous carrier occurs immediately once the carrier material reaches a nozzle position inside the blender.

After impregnation, API-impregnated porous carrier is collected from the continuous impregnation device and solvent is evaporated which leads to the drying of the impregnated porous carrier. Depending on the intended use of the resulting impregnated porous carrier particles, the remaining solvent content can be adjusted. Preferably the evaporation of solvent in step c leads to essentially dry or dry impregnated porous carrier particles. In this context, the term dry refers to a condition of the impregnated porous carrier particles wherein their weight varies less than 1% after being placed in a vented oven at 50° C. for one hour.

A series of subsequent dispensing and mixing steps can be applied, from which the API-impregnated porous carrier may be formulated into a pharmaceutical composition which may be a solid dosage form. Non-limiting examples of solid dosage forms are tablets, capsules, powder blends, and granulates. The finished drug product may be further provided in appropriate packaging, such as but not limited to a blister pack, a bottle, or a vial.

Dissolution testing may be performed to determine the drug release profile of the API-impregnated carrier and also of finished dosage forms manufactured using the API-impregnated carrier. Different carriers, and pharmaceutical excipients such as control release polymers, may be used to impart the API-impregnated carrier, and the products compounded from them, with any desired drug release profile, including, without limitation, immediate release, delayed release, sustained release, pulsed release, etc.

The disclosed continuous impregnation process is usually maintained in a near-steady state condition. In order to get a successful continuous impregnation process, the time window for impregnation is much shorter in a continuous process than in a batch process. In a batch process, there is a set amount of API solution that is dispensed over time. In a continuous process, carrier powder constantly moves through the system. Therefore, the solution addition rate needs to be adjusted to the carrier flow rate to reach a desired output composition ratio of API-impregnated porous material. Hence, in the disclosed continuous impregnation process, it is important to adjust the dispensing rate according to the mean residence time and flow rate of carrier powder from the powder feeder. Also, it is important to determine the maximum allowed dispensing rate, and possibly the impregnator aimpeller RPM (if one is present), to avoid agglomeration of the porous carrier due to local excesses of solution. This step largely depends on the properties of the porous carrier and the throughput of the API material. Those skilled in the art would know how to determine appropriate conditions using routine experiments that examine the effect of varying the aforementioned flow rates and agitation rates.

As used herein, the term “specific surface area” (SSA) is a property of solids defined as the total surface area of a material per unit of mass [m²/g]. According to the invention, the SSA is measured according to DIN ISO 9277:2014-01 by using a “3 Flex Version 3.01-Serial number 324” from Micromeritics Insturment Cooperation, US. The sample is heated up to 250° C. under vacuum. A multi-point measurement is performed with N₂ as carrier gas.

The continuous impregnation process is extremely simple, making it possible to make the finished product by taking a drug solution (often produced during drug synthesis, or alternatively prepared as needed), dispensing it onto a pre-formed porous carrier, drying the dispensed carrier, and filling it into capsules. This process reduces or eliminates the need for very expensive processing steps, including crystallization, drying, milling of the drug material, dry or wet granulation, wet sizing of the wet granulation and drying of the wet granulation. By simplifying the process, the method accelerates product development very significantly. The properties of the impregnated carrier are very similar to those of the un-impregnated carrier, thus providing a generic platform for product development that works for many different drugs. The process enhances water solubility of poorly soluble drugs and is a competitive alternative to hot melt extrusion and to spray drying, which are the much more expensive commercial alternatives currently in use by industry. The process is scalable, facilitating both the manufacture of clinical supplies and its straightforward scale-up to commercial manufacturing scales.

According to the invention, high surface area porous carriers can be impregnated with a high API loading and a high content uniformity within only a few minutes of contact time between the carrier and the impregnation fluid when a continuous impregnation process is used. As used herein, the term “high surface area porous carriers” is used for porous carrier with a specific surface area of at least 400 m²/g. The continuous impregnation process in combination with high surface area carriers has numerous further benefits. The mixing time is significantly shorter, making the system easily scaleable. In addition, if larger amounts of material are desired, this can be achieved by operating the continuous process for a longer period. Moreover, the processes operates at or very close to steady state, where all the flowing material experiences essentially the same processing conditions, thus making it easy to achieve uniform results. In addition, continuous processing equipment is much smaller in size than the batch equipment required to process a similar amount of products, thus enabling large savings in equipment cost and space.

A batch process with the same material may be able to achieve high loadings, but typically requires several hours of contact between the carrier and the API solution. One of the advantages of the high surface area porous carriers is that they enable high loadings while maintaining the enhancement in dissolution of poorly soluble APIs. By enabling high loadings, the current method also reduces the amount of the carrier and other excipients, which leads to a decreased final size of the tablet and makes it easier to swallow. However, such carrier materials are difficult to fluidize and have only been impregnated successfully in batch mode using mechanically agitated vessels that require up to several hours of contact time to achieve homogeneous impregnation. The continuous process disclosed in this patent was surprisingly found to enable homogeneous impregnation with contact times in the order of a few minutes, enabling a much more convenient and consistent process.

Parteck® SLC 500 has been used as a model high surface area porous carriers. Parteck® SLC 500 is a mesoporous silica gel made of silicon dioxide with a specific surface area of 400 to 600 m²/g, in particular approximately 500 m²/g, an average pore size of 1 to 10 nm, in particular 2 to 7 nm, and an average particle size of 5 to 25 μm. It has been found that high API loadings and a high content uniformity can be achieved in less than 2 minutes of contact time when Parteck® SLC 500 is continuously impregnated. Loadings as high as 20% can be obtained using a single pass process, and loadings higher than 35% can be achieved using two passes. This makes it possible to implement a convenient continuous impregnation and drying process that can create a high-loading impregnated carrier, which is therefore suitable for the manufacturing of high dose poorly soluble drugs.

Two compounds, belonging to BCS class II, were used as examples of typical APIs, Ibuprofen and Carbamazepine. As explained above, the disclosed process is not limited to specific APIs as the underlying principle is essentially independent of the physical API characteristics. The disclosed process is also not limited to specific BCS classes. APIs of BCS class II were chosen to show an improvement of the dissolution of the resulting impregnated product.

In a further embodiment of the invention, the porous carrier has a specific surface area of more than 300 m²/g, of at least 400 m²/g, of 400 m²/g to 800 m²/g, 400 m²/g to 600 m²/g, 450 m²/g to 550 m²/g or about 500 m²/g, preferably of at least 400 m²/g.

Non-limiting examples of suitable porous carriers include magnesium aluminum metasilicate, silicon dioxide, dibasic calcium phosphate, calcium hydrogen phosphate (CaHPQ4), porous carriers identified in the Geldart classification, Powder Technology, 7:285-292 (1973) as Group A and/or Group B carriers, and combinations thereof. In some embodiments, the porosity of the carrier in terms of pores by volume may be 20% to 85%, 20% to 70%, 20% to 60%, 20% to 50%, 20% to 40%, 20% to 30%, 30% to 50%, 50% to 70% or 70% to 85%.

The porous carrier may be a pharmaceutically acceptable carrier that is appropriate for use in the intended finished dosage form.

In a further embodiment of the invention, the porous carrier has an average pore size of 1 to 30 nm, 1 to 10 nm or 2 to 10 nm, preferably 1 to 10 nm.

As used herein, the term “average pore size” is a property of porous solids defined as the distance between two opposite walls of the pore (diameter of cylindrical pores). According to the invention the average pore size is measured according to ISO 15901-2:2006. Adsorption- and desorption isotherme were measured using N₂ as adsorbant and calculation of pore size and volume was done according to Barrett, Joyner and Halenda.

In a further embodiment of the invention, the porous carrier has an average particle size of 5 μm to 150 μm, 5 μm to 100 μm, 5 μm to 50 μm, 5 μm to 25 μm or 5 μm to 20 μm, preferably 5 μm to 150 μm.

As used herein, the term “D50”, “median of a particle size distribution”, “average particle size” and “particle size” is a property of solids defined as the diameter in microns where half of the particle population resides above this size, and half resides below this size. The term “median” might refer to half the mass, half the volume, or half the number of particles. For the purposes of this invention, D50 always refers to the volume median, and it is the median for a volume distribution as measured by laser light scattering, using a suitable (wet or dry) method, a suitable obscuration, and settings of the measurement method (e.g. Venturi pressure drop for the dry method) suitable selected so that the measurement is accurate, representative and reproducible. Similarly, 90 percent of volume of the population lies below the D90, and 10 percent of the volume of the population lies below the D10.

According to the invention, the particle size is measured in a particle size analyzer LS 13320 Optical Bench (Beckman Coulter, Inc., NJ USA) using the Tornado Dry Powder System (DPS), and implementing a Venturi pressure drop of 10″ and where the particle size distribution is determined based on the Franhoufer model using a sample size of at least 20 ml and 5% obscuration. In the Tornado DPS the sample is placed in a sample holder and delivered to the sensing zone in the optical bench by a vacuum.

In a further embodiment of the invention, the porous carrier is based on silica oxide porous carrier. In a further embodiment of the invention, the porous carrier is a silicon dioxide porous carrier. In a further embodiment of the invention, the porous carrier is Parteck® SLC 500 (SLC) as defined above.

As shown in Examples 1 and 2, the porous carriers according to the invention have several advantages compared to porous carrier falling outside the ranges as mentioned above. With the inventive continuous impregnation process, the porous carrier according to the invention show a surprisingly superior behavior regarding the following parameters:
Drug loading
Content uniformity
Stabilizing effect/capacity
Particle size distribution after loading
Dissolution performance
Drying time

After two or three impregnation steps, both APIs, Ibuprofen and Carbamazepine, show a higher drug loading on a carrier according to the invention (Parteck SLC 500) compared to porous carrier falling outside the ranges as defined above, e.g. compared to Neusilin and Fujicalin.

Unexpectedly, the content uniformity (measured as RSD in %/C.I. at the significance level of 5%) was also significantly better (e.g., lower RSD) with SLC in all experiments.

Additionally, the porous carriers according to the invention are significantly better in stabilizing the amorphous form of the API compared to the other porous carrier. This is especially apparent with Carbamazepin as API in Example 2. Whereas there is no sign of crystallinity after the second impregnation step with SLC (15.96% drug loading) and only of a minor sign of crystallinity after the third impregnation step with SLC (23% drug loading), there is already a considerable amount of crystalline API detectable after the second impregnation step with Neusilin (12.35% drug loading). Crystalline API is also detectable after the second impregntation step of Carbamazepine on Fujicalin (11.76% drug loading). The stabilization of the API in its amorphous form is crucial to improve its dissolution which is a relevant parameter to enhance the bioavailability of a drug.

In Example 1, no difference was detected regarding the stabilization of the amorphous form of Ibuprofen between SLC and Neusilin. Nevertheless, it is surprising that the API dissolution was faster with SLC as carrier.

With both APIs, Ibuprofen and Carbamazepine, the porous carrier according to the invention shows a faster drug release. A faster drug release (dissolution of an API) enhances the bioavailability of a drug, which is a prerequesite for the effect of a poorly soluble API. In addition, the overall solubility of Carbamazepine is increased using SLC as carrier. This can be seen in Examples 1 and 2 in the dissolution measurements comparing SLC-impregnated carrierer vs. Neusilin-impregnated carrier.

Particle size measurements reveal an overall consistent particle size distribution before and after the impregnation steps for SLC and Neusilin. Fujicalin shows a tendency for agglomeration already after the second loading step with Carbamazepine. Therefore, no third impregnation step was performed with Carbamazepine on Fujicalin. Powder agglomeration during impregantion steps has to be avoided as it can affect processability and drug release.

The flow properties, compaction properties, and particle size distribution of an API-impregnated porous carrier made by the disclosed process are very similar to those of an unloaded carrier, and are largely independent of the API used in the process, thus providing an extremely useful platform for product development that works well for many different drugs. Consequently, APIs having poor or undesirable flow and compaction properties may be impregnated into a porous carrier having improved, desirable or advantageous flow and compaction properties using the continuous manufacturing process disclosed herein, thereby producing API-impregnated particles having predictable properties that are the same or very similar to those of the unimpregnated porous carrier particles. As a result, the disclosed method can produce API-impregnated carriers having excellent flow properties, relatively high bulk density (e.g., higher than 0.5 g/ml), excellent compressibility, and/or minimum tendency to acquire electrostatic charge, mainly by selecting a porous carrier with such properties and then impregnating the API onto that carrier. The disclosed method is also readily up- or down-scalable, facilitating manufacturing at both clinical trial and commercial scales and enabling rapid scale-up (or scale-down) and scale-out of manufacturing rates to meet changing market demands. In its simplest form, a continuous process enables the operator to make as much, or as little product as desired simply by changing the length of time the process is operated.

In a further embodiment of the invention, the content uniformity of the at least one API in the impregnated porous carrier particles is characterized by a relative standard deviation (RSD) of less than or equal to 5%, less than or equal to 4% less than or equal to 3%, less than or equal to 2%, less than or equal to 1% or less than or equal to 0.5% when tested using samples of at least 400 mg of the impregnated porous carrier particles. In general, the lower the value of the RSD, the more homogeneous the API distribution on the material and the overall material.

As used herein, the term “content uniformity” quantifies the homogeneity of the API distribution across the volume of the porous carrier, for a given size of samples, similar to the amount of impregnated carrier to be used in a finished dosage form. According to the current invention the term is used synoymuously with the term “uniformity”, “homogenuous API distribution”, “homogeneity of API distribution” and “blend uniformity”.

The disclosed methods, if properly implemented and aided by routine experiments to select suitable values of varying process parameters such as dispense rate, impeller rates, fluid temperatures, etc., can and will achieve a highly uniform product. The homogeneity of the at least one API in the impregnated porous carrier particles is characterized by the relative standard deviation (RSD) of the content of the API in a cohort of impregnated porous carrier samples. The desirable value of the RSD is less than or equal to 5%, preferably less than or equal to 3%, more preferably less than or equal to 2%, most preferably less than or equal to 1% when tested using samples of at least 400 mg of the impregnated porous carrier particles, in particular for blends containing any amount of API from 0.1 wt % to 40 wt %, preferably 1 wt % to 30 wt %, more preferably 5 wt % to 20 wt %.

This is not known to be achieved by batch impregnation processes. At a comparable API loading of the impregnated porous carrier, the homogeneity of API distribution is characterized by a higher relative standard deviation (RSD) in batch processes. This is also not known to be achieved by continuous impregnation processes with porous carrier falling outside the range of the parameters mentioned in present invention, e.g. specific surface area and/or average particle size and/or average pore size. Especially, the combination of a high API loading of the impregnated porous carrier particles within a relatively short contact time between the porous carrier and the solution containing at least one API, which is possible with the porous carrier of the present invention, is not known to be archieved by continuous impregnation processes of carrier materials with lower surface areas.

In a further embodiment of the invention, the API loading of the impregnated porous carrier particles is higher compared to the API loading of impregnated porous carrier particles with a porous carrier having a specific surface area of less than 400 m²/g, of less than 400 m²/g and an average pore size outside the range of 1 to 10 nm or of less than 400 m²/g and an average particle outside the range of 5 μm to 150 μm, of less than 400 m²/g and an average pore size outside the range of 1 to 10 nm and an average particle outside the range of 5 μm to 150 μm.

In a further embodiment of the invention, the API loading of the impregnated porous carrier particles is higher than 10%, 15%, 20%, 25%, 30%, 40% or 50%.

In a further embodiment of the invention, the API release of the impregnated porous carrier particles is faster compared to the API release of impregnated porous carrier particles with a porous carrier having a specific surface area of less than 400 m²/g, of less than 400 m²/g and an average pore size outside the range of 1 to 10 nm, less than 400 m²/g and an average particle outside the range of 5 μm to 150 μm, of less than 400 m²/g and an average pore size outside the range of 1 to 10 nm and an average particle outside the range of 5 μm to 150 μm. In a further embodiment the API release refers to the release of API from the solide dosage form. In a further embodiment the API release refers to the release of 50% API in less than one hour from the impregnated porous carrier particles or the solid dosage form.

In a further embodiment of the invention, the API release of the impregnated porous carrier particles is faster compared to the API release of the identical impregnated porous carrier being manufactured by batch impregnation process.

Dissolution testing is performed to determine the release of the API from the impregnated porous carrier and also of the solid dosage forms manufactured using the API-impregnated carrier. According to the invention the release of API is performed using a USP dissolution apparatus type II (paddle). Preferably a Sotax AT Extend (Sotax AG, Lörrach, Germany) equipped with a photometer (Specord 200+, Analytik Jena AG, Jena, Germany) is used, even more preferably the dissolution test is performed in simulated gastric fluid (SGFsp) at 37° C. over 120 min at 75 rpm paddle speed.

In a further embodiment of the invention, the continuous impregnation device is a continuous tubular mixer, a vertical continuous conical mixer, a continuous ribbon blender, a continuous rotating/tumbling mixer, a twin-screw processor or a continuous sigma blade mixer, or an equivalent device performing the same function in the same way to achieve the same result. Preferably, the continuous impregnation device is a continuous tubular mixer equipped with an axial agitator.

The evaporation of solvent in step c refers to a drying step of the impregnated porous carrier. The process can be a continuous evaporation or a batch evaporation step. According to the invention the terms “evaporation”, “evaporating solvent” and “drying” are used synonymously. A continuous evaporation can be carried out at least in part in a continuous dryer, selected from a group consisting of a continuous fluid bed dryer, a heated screw conveying device, a belt oven, a vibratory conveyor, and a continuous rotary dryer.

A batch evaporation step can be carried out at least in part in a batch dryer, selected from a group consisting of a batch fluid bed processor, a batch oven, a batch convective mixer, and a batch tumbling dryer. The drying of the impregnated porous carrier can take place at least partially within the impregnation device. In a further embodiment of the invention, at least 10% of the solvent evaporates in a separate continuous dryer.

Surprisingly the drying time for the inventive continuous impregnation process with the porous carrier as mentioned above, in particular SLC, is shorter as compared to carriers falling outside the ranges as defined above, e.g. compared to Neusilin and Fujicalin. A shorter drying time is especially advantageous for the manufacture of solid dosage forms in a commercial scale as the manufacturing process is faster and more cost-effective.

In a further embodiment of the invention, the mean residence time of the porous carrier in the continuous impregnation device is less than 20 minutes, less than 10 minutes, less than 5 minutes, less than 3 minutes, less than 2 minutes, or less than 1 minute.

In a further embodiment of the invention, the residence time of the porous carrier in the continuous impregnation device is lower than the residence time of the porous carrier in a batch impregnation.

As used herein, the term “residence time”, “mean residence time” or “MRT” is the arithmetic average of the residence time distribution of the carrier in the continuous impregnation device. According to the present invention, the residence time is measured according to the methods disclosed in (i) Muhr H, Leclerc J. P, and David R. Fluorescent UV dye: A particularly well-suited tracer to determine residence time distributions of liquid phase in large industrial reactors. Analusis. 1999; 27:541-543 and (ii) Engisch W, Muzzio F. Using Residence Time Distributions (RTDs) to Address the Traceability of Raw Materials in Continuous Pharmaceutical Manufacturing. J. Pharm. Innov. 2016; 11: 64-81.

In a further embodiment of the invention, the sequence of steps a, b and c is applied only once. In a further embodiment of the invention, the sequence of steps a, b and c is applied 2 or more times.

In a further embodiment, the dry impregnated porous carrier particles are mixed with at least one pharmaceutical excipient to form a pharmaceutical composition. As used herein, the term “pharmaceutical composition” refers to the mixture of the impregnated porous carrier particles and at least one pharmaceutical excipient. As used herein, the term “pharmaceutical excipient” includes carriers, such as cellulose or substituted cellulose materials, sodium citrate or dicalcium phosphate; fillers or extenders, such as starch-based materials, microcrystalline cellulose, lactose, sucrose, glucose, mannitol, and/or silicic acid; binders, such as hydroxypropyl cellulose, hydroxypropyl methylcellulose, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; absorption accelerators, such as quaternary ammonium compounds and surfactants, such as poloxamer and sodium lauryl sulfate; wetting agents, such as cetyl alcohol, glycerol monostearate, and non-ionic surfactants; absorbents, such as kaolin and bentonite clay; lubricants, such as talc, calcium stearate, magnesmm stearate, solid polyethylene glycols, sodium lauryl sulfate, zmc stearate, sodium stearate, stearic acid, and mixtures thereof; coloring agents; controlled release agents, such as crospovidone, ethyl cellulose, poly(ethylene oxide), alkyl-substituted celluloses, crosslinked polyacrylic acids, xanthan gum, guar gum, carrageenan gum, locust bean gum, gellan gum, methyl cellulose, hydroxypropyl methyl cellulose, hydroxyethyl cellulose, ethyl cellulose, sodium carboxymethyl cellulose, sodium alginate, gelatin, modified starches, co-polymers of carboxyvinyl polymers, co-polymer of acrylates, co-polymers of oxyethylene and oxypropylene and mixtures thereof; and other additives, such as paraffin and high molecular weight polyethylene glycols. In mentioning these materials, the use of the term “such us” means that the mentioned materials are simply examples belonging to a more extensive class. Also, as it is known in the art, most pharmaceutical ingredients have more than one useful function, and the mention of any one ingredient in any one of the above examples is not meant to exclude the use of that material for a different purpose. For example, starch can be a filler, a binder, and a disintegrant, and many other materials can also be used in more than one way. Usage of these materials to achieve expected outcomes is well described in the art and it is not, by itself, to be considered inventive.

Preferably the at least one pharmaceutical excipient is selected from the group comprising a filler, a disintegrant, a modified release agent, a surfactant, and a lubricant, to form a pharmaceutical composition.

In a further embodiment of the invention, the present invention discloses a pharmaceutical composition obtainable or obtained by a process mentioned above.

In some embodiments, the disclosed technology relates to processes of continuously manufacturing a solid dosage form using an API-impregnated porous carrier. The step for processing the impregnated porous carrier particles into a solid dosage form can by either a continuous or a non-continuous (e.g., batch) process. In such embodiments, the material being processed in the continuous process flows through multiple simultaneously active unit operations, including feeding API-impregnated porous carrier using a feeder, optionally combining the API-impregnated porous carrier with one or more pharmaceutically acceptable excipients in a blender, and compounding the mixture into a desired solid dosage form. Nonlimiting examples of such formulating steps include filling the mixture into capsules, vials, sachets, or aerosol blisters, or compressing the mixture into tablets.

In a further embodiment, the impregnated porous carrier particles or the pharmaceutical composition are processed into a solid dosage form.

As used herein, the term “solid dosage form” refers to compositions that are suitable for administration to a subject, such as a human or other mammal. Non-limiting examples of solid oral dosage forms include tablets, capsules containing the impregnated carrier possibly together with other ingredients, capsules comprising a plurality of mini-tablets, powders, and granulations. Non-limiting examples of tablets include monolith tablets (coated or uncoated), sublingual molded tablets, buccal molded tablets, sintered tablets, compressed tablets, chewable tablets, freeze-dried tablets, soluble effervescent tablets, lozenges, and implants or pellets. Non-limiting examples of capsules, in which the composition is enclosed within a hard or soft soluble container or shell, include hard gelatin capsules, soft gelatin capsules, and non-gelatin capsules. In some embodiments, the finished solid oral dosage form may be modified to achieve a desired timing of API release—e.g., a dosage form that provides immediate release, sustained release, controlled release, extended release, partial immediate and partial delayed release, and combinations thereof. The disclosed methods can also be used in the manufacture of non-oral products where a mixture of APis and other solid ingredients is useful, including but not limited to the manufacture of inhalants, implantable and injectable solid compositions, vascular stents, and the like. While other types of porous materials might be needed in the formulation of such products, the inventive concepts disclosed here can be used in combination with routine experiments to implement methods and processes applicable to such products. Preferably, the solid dosage form is a capsule or a tablet.

In a further embodiment of the invention, the present invention discloses a solid dosage form obtainable or obtained by a process mentioned above.

In a further embodiment of the invention, the present invention discloses impregnated porous carrier particles obtainable or obtained by a continuous impregnation process as mentioned above.

The impregnated porous carrier particles can have an API content that is less than 1%, less than 5%, less than 10%, less than 20%, less than 30%, less than 40% or less than 50% by weight.

In a further embodiment of the invention, the impregnated porous carrier particles have a particle size distribution with a volume-based D90 that differs by less than 20% from the D90 of the porous carrier ingredient, as measured by Laser light scattering in a LS 13320 Optical Bench (Beckman Coulter, Inc., NJ USA).

According to the invention, the particle size distribution is measured by Laser light scattering in LS 13320 Optical Bench (Beckman Coulter, Inc., NJ USA).

In a further embodiment of the invention, the impregnated porous carrier particles have minor and major principal stresses that differ by less than 20% from the minor and major principal stresses of the porous carrier ingredient, as measured in the FT4 freeman technology rheometer using a 50 ml probe and a normal load of 3 KPa. The procedure includes four steps: conditioning, consolidation, pre-shearing, and shearing.

As used herein, the term “minor and major principal stresses” are determined from the Mohrs diagram of the yield Locii of the material at the recited normal load.

### EXAMPLES

The disclosed technology is next described by means of the following non-limiting examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the claims, along with the full scope of equivalents to which the claims are entitled. Efforts have been made to ensure accuracy with respect to values presented (e.g., amounts, temperature, etc.), but some experimental error and deviation should be accounted for. Unless indicated otherwise, parts are parts by weight, molecular weight is weight average molecular weight, particle size distribution is volume-based particle size distribution, temperature is in degrees Centigrade, and pressure is at or near atmospheric.

Table 1 is showing the properties of the three carriers Fujicalin, Neusilin US2 and Parteck SLC 500.

TABLE 1

| Properties | Fujicalin | | Neusilin US2 | | Parteck SLC 500 | |
|---|---|---|---|---|---|---|
| Product | Spherically | | Granulated | | Non-ordered | |
| | granulated | | synthetic, | | mesoporous | |
| | dicalcium | | amorphous form of | | silica (6) | |
| | phosphate | | magnesium | | | |
| | anhydrous (1) | | aluminometasilicate (3) | | | |
| D50 [μm] | 120-130 | (7) | 105-125 | (7) | 13.9-23 | (7) |
| Specific surface | 28.7 | (7) | 390.4 | (7) | 471 | (7) |
| area [m²/g] | | | | | | |
| Pore Size [nm] | 7.35 | (1) | 18.76 | (5) | 6-10 | (5) |
| | | | Broad pore size | | Sharp pore size | |
| | | | distribution (5) | | distribution (5) | |
| Pore Volume | 0.2242e−01 | (7) | 1.742 | (7) | 0.83 | (5) |
| [cm³/g] | | | | | | |
| Bulk Density | 0.431 | (2) | 0.17 | (4) | 0.312 | (7) |
| [g/cm³] | | | | | | |
| Tapped Density | 0.484 | (2) | 0.19 | (4) | 0.485 | (7) |
| Hausner Ratio | 1.123 | (2) | 1.15 | (4) | 1.55 | (7) |

(1) Takami K, Machimura H, Takado K, Inagaki M, Kawashima Y. Novel Preparation of Free Flowing Spherically Granulated Dibasic Calcium Phosphate Anhydrous for Direct Tabletting. Chem. Pharm. Bull. 1996; 44(4): 868-870
(2) Schlack H, Bauer-Brandl A, Schubert R, Becker D. Properties of Fujicalin, a new modified anhydrous dibasic calcium phosphate for direct compression: Comparison with dicalcium phosphate dihyradte. Drug Dev Ind Pharm. 2001; 27 (8): 789-801
(3) https://www.fujichemical.co.jp/english/medical/medicine/neusilin/neusilin_brochure.pdf
(4) Gumaste, S. G.; Dalrymple, D. M.; Serajuddin, T. M. Development of Solid SEDDS, V: Compaction and Drug ReleaseProperties of Tablets Prepared by Adsorbing Lipid-Based Formulations onto Neusilin@US2. Pharm. Res. 2013, 30, 3186-319
(5) Barbora V, Andreas N, Zdenka S, Martin K. Relevance of the theoretical critical pore radius in mesoporous silica for fast crystallizing drugs. International Journal of Pharmaceutics 591 (2020)
(6) A.-L. Lainé, D. Price, J. Davis, D. Roberts, R. Hudson, K. Back, P. Bungay, N. Flanagan, enhanced oral delivery of celecoxib via the development of a super saturable amorphous formulation utilizing mesoporous silica and co-loaded HPMCAS, Int. J. Pharm. 512 (2016) 118-125.
(7) Internal mesaurements according to the methods as shown below

### Example 1 (Continuous Impregnation of IBU on SLC and NEU)

This example describes an impregnation process achieving high drug load using a continuous blender and Ibuprofen as model drug. As porous carrier Parteck® SLC 500 and Neusilin US2 as defined above were used.

### Measurements:

Drug load
Content uniformity
XRD (Crystallinity)
Particle size distribution
Dissolution

The example describes one embodiment of the disclosed continuous processing for manufacturing pharmaceuticals using continuous impregnation of API onto a porous carrier. As porous carrier Parteck® SLC 500 (Merck KGaA, Darmstadt) (SLC) and Neusilin US2 (NEU) were used.

The API used in this example was Ibuprofen (IBU). Ibuprofen was used for impregnation and also as a liquid phase tracer in residence time distribution (RTD) measurements.

### Impregnation Process

The equipment used in this study included a Glatt continuous powder blender-GCG-70 (Glatt® group, Binzen, Germany), which was used as the liquid-solid contactor device, a single K-Tron K-CLSFS KT20 (Coperion K-Tron Pitman Inc., NJ) gravimetric feeder for manufacturing of impregnated carrier porous particles, and a FT-NIR Matrix (Bruker Optics Billerica, MA, USA) for spectral acquisition to study RTD and relative standard deviation (RSD) of impregnated products. The continuous powder blender was operated with a standard blending shaft at 150 revolutions per minute (rpm), using the standard Glatt blade configuration.

Analytical grade methanol (MeOH) was used as the transport solvent, to dissolve the Ibuprofen, and dispense it into the impregnation device. The continuous impregnation process included multiple unit operations and online testing equipment including a loss-in-weight (LIW) feeder, a peristaltic pump, a continuous blender, a near infrared (NIR) spectroscopy instrument, and a vibratory feeder.

First, the porous carrier was fed through the LIW feeder at a flow rate of 6 kg/h into the blender, where the impregnation step occurred immediately once the material reached the nozzle position inside the blender. A peristaltic pump was used to dispense the drug solution through tubing connected to the 0.1 mm diameter nozzle, the pump rate was 40 g/min, the Ibuprofen concentration (dissolved in MeOH) was 50% w/w. Next, the impregnated particles were dried using an oven at 45° C. for three days before reimpregnation and at 40° C. for 5 days before characterization, to completely evaporate the methanol (SLC-IBU (1X) and NEU-IBU (1X)). A second impregnation step was applied at 6 kg/h and with a 45% Ibu/MeOH solution, dispensed at 40 g/min (SLC-IBU (2X) and NEU-IBU (2X)). Process results are shown in table 2 with 1 kg of carrier. It is to be noticed that the drying time for NEU (not less than 30 hours) was considerably longer than for SLC (not less than 24 hours).

TABLE 2

| | Run 1 | Run 2 |
|---|---|---|
| Loading time | Less than 6 minutes | Less than 6 minutes |
| Drying time | Not less than 24 hours | Not less than 24 hours |

### Measurement of Drug Load and Content Uniformity

For quantification of Ibuprofen, 2.0 g of the loaded powder were transferred to a test tubes and 10.0 ml of methanol were added. A mechanical agitator was used for 30 s to dissolve the ibuprofen. The test tubes were subjected to centrifugation at 4000 rpm during 100 min. A reverse phase column using methanol as mobile phase was used. Flow rate was 1 ml/min and UV detection at a wavelength of 219 nm. The ibuprofen injection amount was 20 μl. The stoptime was 5 mins and the post time was 1 min. The retention time of ibuprofen is 1.7 minutes.

The content uniformity was then assessed by calculating the relative standard deviation of the n=10 drug load results (sampling˜500 mg each). Results can bee found in table 3.

TABLE 3

| Product | Average % | RSD % | C.I. |
|---|---|---|---|
| IBU-SLC-500 2X | 35.90 | 0.801 | 0.497-1.66 |
| IBU-NEU 2X | 26.11 | 4.516 | 2.806-9.387 |

### Measurement of XRD (Crystallinity)

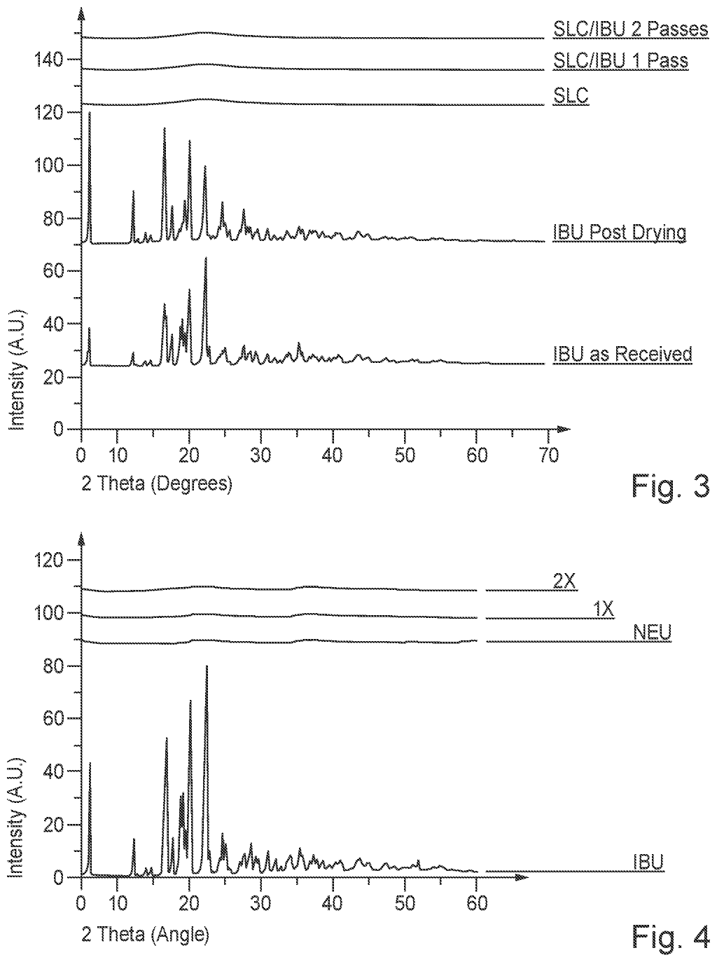

Powder X-ray diffraction (p-XRD) was used to determine the physical state of the drug. P-XRD patterns were obtained using a PANalytical X'pert, which was operated at 35 kV and 50 mA. The scan procedure included the following conditions: scan axis, gonio; scan range (°), 5-70; step size (°), 0.0131; scan mode: continuous; counting time (s), 4.845. The model drug was in an amorphous state after each loading step (“1 Pass” and “2 Passes”) as shown in FIGS. 3 and 4. “IBU as received” (pure, particulate model drug) and “IBU Post Drying” (model drug dissolved in MeOH and recrystallized again) serve as references.

### Measurement of Particle Size Distribution

Particle size distribution was measured in a particle size analyzer LS 13320 Optical Bench (Beckman Coulter, Inc., NJ USA) using the Tornado Dry Powder System (DPS), and implementing a Venturi pressure drop of 10″ and where the particle size distribution is determined based on the Franhoufer model using a sample size of at least 20 ml and 5% obscuration. In the Tornado DPS The sample is placed in a sample holder and delivered to the sensing zone in the optical bench by a vacuum. Results are shown in table 4.

TABLE 4

| Product | D10 (μm) | D50 (μm) | D90 (μm) | Mean (μm) |
|---|---|---|---|---|
| SLC | 2.38 | 23.77 | 51.39 | 25.53 |
| IBU-SLC-500 2X | 5.83 | 27.27 | 51.87 | 27.94 |
| NEU | 42.65 | 116.10 | 190.08 | 116.15 |
| IBU-NEU 2X | 45.92 | 119.00 | 192.3 | 119.05 |

### Measurement of Dissolution

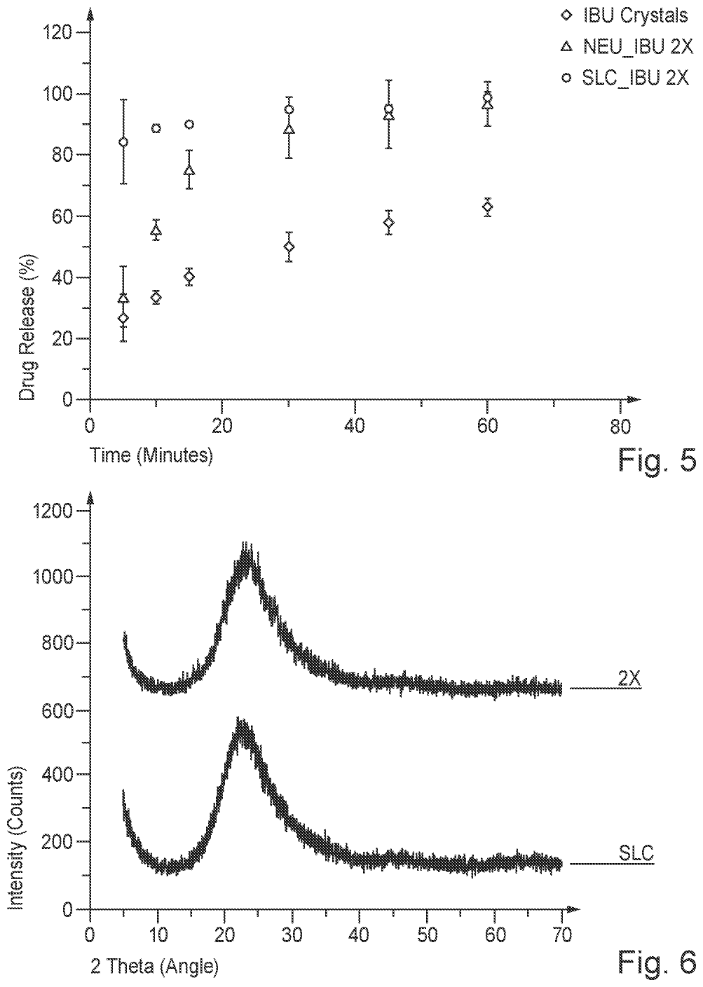

The dissolution profiles of ibuprofen powder and impregnated products of SLC-IBU (2X) and NEU-IBU (2X) were studies using a 708-DS, 8-spindle, 8-vessel USP dissolution equipment type I (basket). Approximately 30 mg of IBU powder and 100 mg (equivalent to about 30 mg of IBU powder) of the impregnated products were hand-filled into hard gelatin capsules (Size 1). The experimental conditions were set up as the following: 0.1 N HCl (500 mL) dissolution medium; 150 rpm agitation; 220 nm UV detection; 37° C. temperature; 5-minute, 10-minute, 15-minute, 30-minute, 45-minute, and 60-minute sample intervals. The samples were manually collected using a syringe equipped with dissolution cannulas and filters. UV spectroscopy of the samples was then measured to determine the percentage of drug release over time. The dissolution tests were undertaken in triplicate. Results of the dissolution experiments can be found in FIG. 5.

### Example 2 (Continuous Impregnation of Carba on SLC, NEU and FUJ)

This example describes an impregnation process achieving high drug load using a continuous blender and Carbamazepine (Carba) as model drug. As porous carrier Parteck® SLC 500 (SLC), Neusilin US2 (NEU), Fujicalin (FUJ) as defined above were used.

### Measurements:

Drug load
Content uniformity
XRD (Crystallinity)
Particle size distribution
Dissolution

The impregnation process was performed according to the method described in Example 1, except for Carbamazepine concentration in the solvent methanol which was 20%.

### Measurement of Drug Load and Homogeneity

Drug loading and blend uniformity were measured using UV/visible spectroscopy (Varian Cary 50 Bio, Agilent Technologies, USA). Ten samples were taken from the resulting products. Four hundred milligrams from each sample was placed in 100 ml conical flasks; and methanol was added till the 100 ml mark. This dispersion was sonicated for 40 min and left overnight at room temperature to ensure a complete extraction of Carbamzepine from the porous carrier. Samples were then withdrawn from each flask using a millipore (10 micron) syringe filter and dilution was done (if necessary). The UV readings at A=285 nm were measured to quantify the amount of carmbmazepine in each sample.

In order to quantify the measured absorbance (amount of Carba in each sample), a calibration curve was constructed by measuring the UV absorbance of carba in methanol at five different concentrations. Therefore, the carba loading of each sample, the mean, the standard deviation, and the percent relative standard deviation (percent RSD) for each batch were calculated. Calculation of % RSD's confidence intervals (C.I.)

The confidence intervals of the resulting % RSD were calculated using [Reference: Gao, Y., Ierapetritou, M. G. & Muzzio, F. J. Determination of the Confidence Interval of the Relative Standard Deviation Using Convolution. J Pharm Innov 8, 72-82 (2013).

$$v ∈ ( \sqrt{\frac{( n - 1 ) {V}^{2}}{{( χ / μ )}_{1 - α / 2 , n - 1}^{2}}} , \sqrt{\frac{( n - 1 ) {V}^{2}}{{( χ / μ )}_{α / 2 , n - 1}^{2}}}$$

where v is the confidence interval; V is the expected value of the RSD measurement; n−1 is the degree of freedom; (χ/μ) 2 is chi-on-mu-square, which is a statistical value, that can be obtained from a table for different significance levels and degrees of freedom.

As shown in Table 5, RSD vale % for SLC is 2.2 (2X) and 3.0 (3X), which indicates a very homogeneous product. The intervals of confidence are fully comprised within 0% to 6% indicating a high level of assurance regarding the homogeneity of the samples. For all remaining experiments, the C.I.s are outside this range, indicating that the blend is mure likely to fail a blend homogeneity assessment.

TABLE 5

| | Average | | C.I. at the |
|---|---|---|---|
| | Drug | RSD, | significance |
| Product | Loading, % | % | level of 5% |
| SLC-Carba-2X | 15.96 | 2.2 | 1.43-4.09 |
| SLC-Carba-3X | 22.89 | 3.0 | 1.95-5.58 |
| NEU-Carba-2X | 12.35 | 4.8 | 3.12-8.93 |
| NEU-Carba-3X | 27.58 | 13.3 | 8.66-24.75 |
| FUJ-Carba- 2X | 11.76 | 8.95 | 5.82-16.65 |

### Measurement of XRD (Crystallinity)

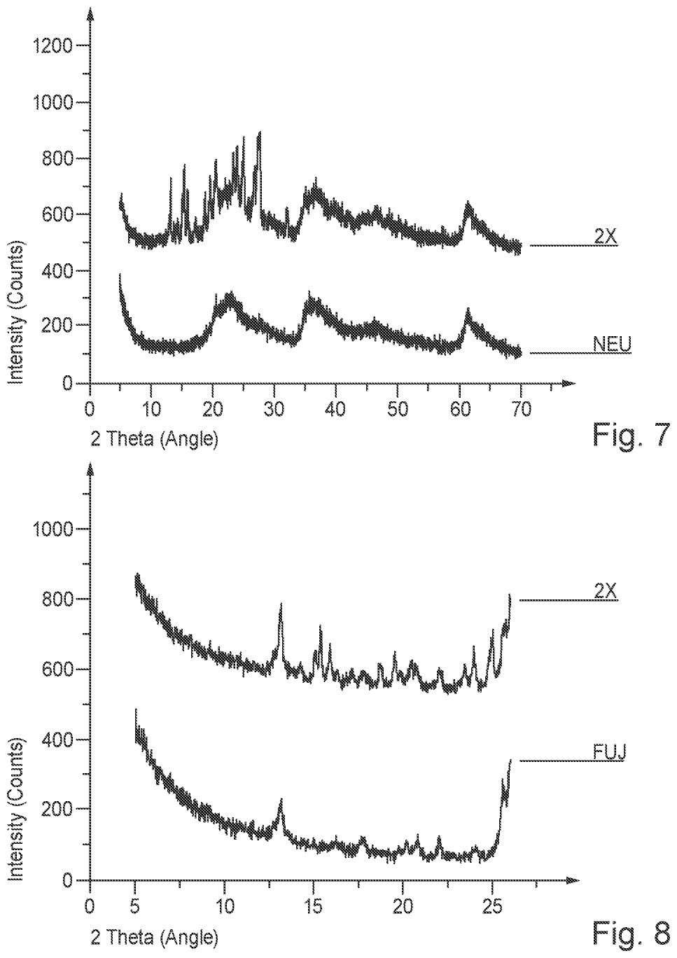

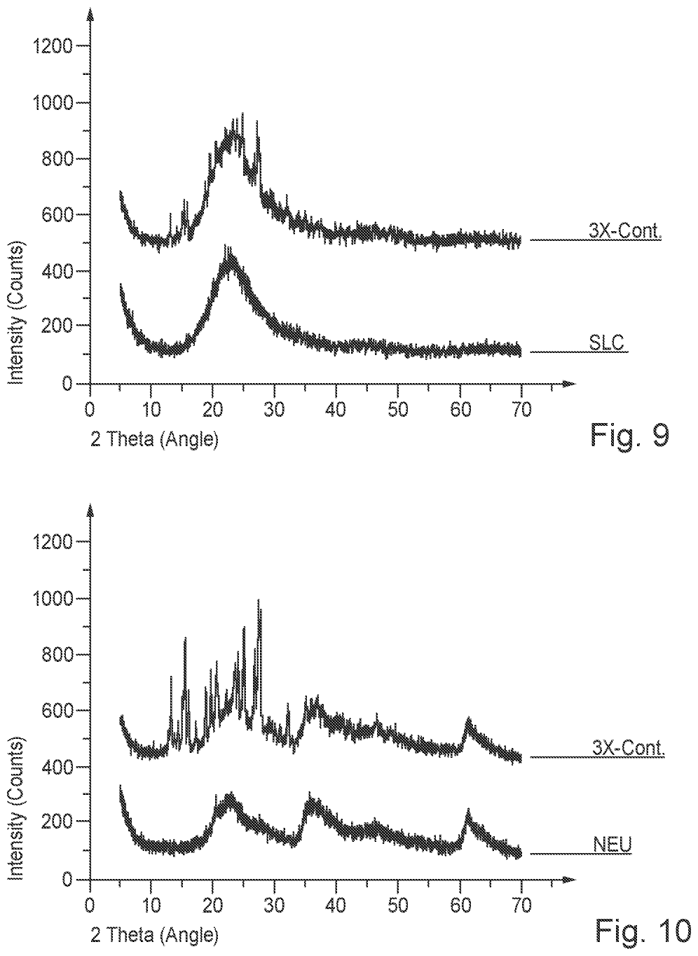

Powder XRD was performed using the method as described in Example 1. FIGS. 6 to 8 show XRPD diagrams for two-time impregnated SLC (FIG. 6), NEU (FIG. 7) and FUJ (FIG. 8) with Carbamazepine and the pure carriers as reference. FIGS. 9 and 10 show XRPD diagrams for three-time impregnated SLC (FIG. 9) and NEU (FIG. 10) with Carbamazepine and the pure carriers as reference.

### Measurement of Particle Size Distribution

Particle size distribution of Carbamazepine-loaded carrier were measured using the method as described in Example 1. Results shown in Table 6 indicated that the PSD of the SLC and NEU carrier and SLC and NEU-impregnated product matches meaning that there is no API on the external particle surface, no fines and no agglomerates. The D90 of FUJ-Carb-2x is significantly elevated compared to FUJ-Carb-1x, suggesting an agglomeration of carrier particles.

TABLE 6

| Product | D10 (μm) | D50 (μm) | D90 (μm) |
|---|---|---|---|
| Pure SLC | 2.38 | 23.77 | 51.39 |
| SLC-Carba-1x | 2.20 | 23.73 | 50.8 |
| SLC-Carba-2x | 2.09 | 23.28 | 50.79 |
| SLC-Carba-3x | 2.23 | 26.07 | 53.41 |
| Pure NEU | 42.65 | 116.10 | 190.08 |
| NEU-Carba-1X | 44.17 | 118.57 | 191.73 |
| NEU-Carba-2X | 41.27 | 118.5 | 194 |
| NEU-Carba-3X | 44.63 | 122.87 | 188.2 |
| Pure FUJ | 50.45 | 130.51 | 187.30 |
| FUJ-Carba-1x | 21.30 | 132.5 | 186.46 |
| FUJ-Carba-2x | 21.87 | 131.21 | 307.12 |

### Measurement of Dissolution

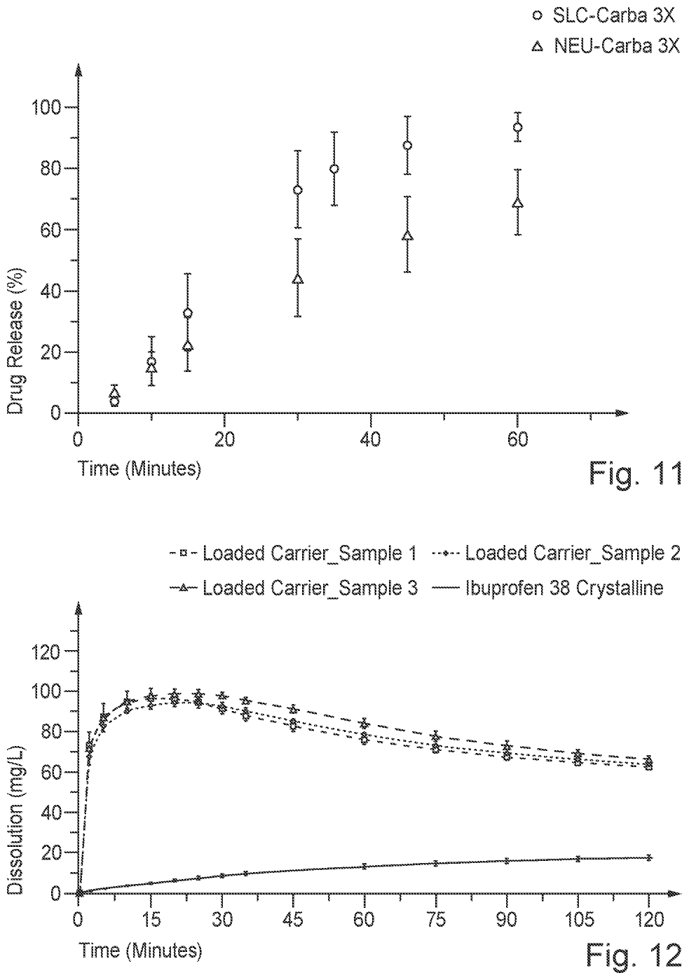

The dissolution profiles of carbamazepine from the impregnated products SLC-Carba (3X) and NEU-Carba (3X) were tested using a 708-DS, 8-spindle, 8-vessel USP dissolution apparatus type II (paddle), with automated online UV-Vis measurement (Agilent Technologies). Dissolution tests were conducted on hard gelatin capsules, which were prepared from the impregnated products. The experimental conditions were set up as the following: dissolution medium: water containing 1% sodium lauryl sulphate; agitation speed: 75 rpm; UV detection: A=288 nm; temperature: 37° C.; 5-minute, 10-minute, 15-minute, 30-minute, 45-minute, and 60-minute sample intervals. The samples were automatically collected and the dissolution tests were undertaken in triplicate. Results of the dissolution experiments can be found in FIG. 11.

### Example 3 (Batch Impregnation of Ibu on SLC)

This example describes an impregnation process using a batch blender and Ibuprofen as model drug. As porous carrier 0.75 kg Parteck® SLC 500 as defined above was used.

The equipment used in this study included a Magic Plant from IKA®-Werke GmbH & Co. KG with a two-fluid nozzle (nozzle orifice 0.6 mm) and a condenser set (about 0.8 bar vacuum). The vessel was heated via the double jacket (60° C.). A low level nitrogen sweep was applied. The tip speed was 0.4 m/s.

Analytical grade acetone (0.75 g) was used as the transport solution, to dissolve the Ibuprofen (0.25 g), and apply it to the carrier. The maximum feed flow rate was 0.6 g/min in order to avoid agglomerates.

The drying of the loaded carrier was performed at 60° C. (jacket temperature), 0.5 bar vacuum for 6 hours in the Magic Plant equipment. The agitation was kept constant at 13 rpm.

For quantification, NMR was used. About 30 mg was weighed and dissolved in deuterated DMSO and analyzed using a Bruker Avance III, 500 MHZ NMR spectrometer (Bruker Corporation, Billerica, USA). Maleic acid was used as internal standard. Measurements were done in triplicate. For content uniformity, the relative standard deviation was calculated of the drug. Table 9 is showing different specifications of the process and the impregnated porous carrier. Table 10 refers to the particle size distribution of the carrier before and after impregnation.

TABLE 9

| | | |
|---|---|---|
| Loading process time | 6:01 | h |
| Drying time | ~23 | h |

| | |
|---|---|
| Yield | 89.4% |
| Drug Load | 30.8% |

TABLE 10

| PSD | Pure SLC | Loaded carrier |
|---|---|---|
| D10 | 2.1 | 2.9 |
| D50 | 13.9 | 29.2 |
| D90 | 29.5 | 75.0 |

Differential scanning calorimetry was used to evaluate the amorphous state on a Mettler Toledo DSC 821e (Mettler Toledo, Gießen, Germany). Nitrogen was used to purge the thermal analysis system (50.0 mL/min). Around 7 mg of loaded carrier was weighed. The temperature range was 30-100° C. and a heat rate of 5 K/min was applied. Three samples were analysed. The results (FIG. 8) confirm the amorphous state of the drug loaded on the porous silica carrier.

Drug release tests of the loaded powder were performed according to USP Paddle method using a Sotax AT Extend (Sotax AG, Lörrach, Germany) equipped with a photometer (Specord 200+, Analytik Jena AG, Jena, Germany). 1000 mL simulated gastric fluid (SGFsp) was used as medium at 37° C. Experiments were done over 120 min at 75 rpm paddle speed. Detection wavelength was 221 nm. Sampling amount of drug loaded carrier was around 167 mg, corresponding to around 50 mg of pure drug (non-sink conditions). Results are depicted in FIG. 12.

1. A continuous impregnation process for making impregnated porous carrier particles, comprising the steps
a. continuously dispensing a solution containing at least one API and feeding a porous carrier in a controlled ratio into a continuous impregnation device,
b. continuously mixing and conveying the drug solution and the porous carrier in the continuous impregnation device and
c. evaporating solvent to form impregnated porous carrier particles,
wherein the porous carrier has a specific surface area of at least 400 m²/g.

2. A process according to claim 1, wherein the porous carrier has an average pore size of 1 to 10 nm.

3. A process according to claim 1, wherein the porous carrier has an average particle size of 5 μm to 150 μm.

4. A process according to claim 1, wherein the porous carrier is a silicon dioxide porous carrier.

5. A process according to claim 1, wherein the content uniformity of the impregnated porous carrier particles is characterized by a relative standard deviation (RSD) of less than or equal to 5% when tested using samples of at least 400 mg of the impregnated porous carrier particles.

6. A process according to claim 1, wherein the API loading of the impregnated porous carrier particles is higher compared to the API loading of impregnated porous carrier particles with a porous carrier having a specific surface area of less than 400 m²/g.

7. A process according to claim 1, wherein the continuous impregnation device is a continuous tubular mixer.

8. A process according to claim 1, wherein step c is a continuous evaporation.

9. A process according to claim 1, where the mean residence time of the porous carrier in the continuous impregnation device is less than 10 minutes.

10. A process according to claim 1, where the impregnated porous carrier particles are mixed with at least one pharmaceutical acceptable excipient to form a pharmaceutical composition.

11. A process according to claim 1, where the impregnated porous carrier particles or the pharmaceutical composition are processed into a solid dosage form.

12. Impregnated porous carrier particles obtainable by a continuous impregnation process according to claim 1.

13. Impregnated porous carrier particles according to claim 12, wherein the content of the at least one API is less than 50% by weight.

14. A pharmaceutical composition obtainable by a process according to claim 10.

15. A solid dosage form obtainable by a process according to claim 11.
